(19) United States
(12) Patent Application Publication
(10) Pub. No.: US 2005/0196826 A1
(43) Pub. Date: Sep. 8, 2005

(54) Self calibrating detection

(76) Inventors: James Vig Sherrill, Oak Ridge, TN (US); C. Douglas Yeager, Knoxville, TN (US)

Correspondence Address: LUEDEKA, NEELY & GRAHAM, P.C., P O BOX 1871, KNOXVILLE, TN, 37901, US

(21) Appl. No.: 10/794,118

(22) Filed: Mar. 5, 2004

(51) Int. Cl. G06F 19/00; G01N 33/48; G01N 33/50; G01N 1/30; G06K 9/00; C12M 1/34

(52) U.S. Cl. 435/40.5; 435/287.1; 702/19; 382/128

(57) ABSTRACT

The present invention is an apparatus and method for calibrating a slide reader which reads probes, such as a biological slide reader. The apparatus has two matched lasers which are directed onto a non-fluorescing probe. Fluorescence from that probe is detected and signals are produced which represent that detection. The signals are compared using either a difference method or a percentage method, and a calibration factor is calculated. A plurality of iterations of the above procedure using a plurality of non-fluorescing probes may be used in order to calculate a more accurate calibration factor. Test probes on the slide are designed to fluoresce when exposed to a target (such as a virus or bacteria or other type of matter) and to electromagnetic radiation of a proper frequency. When adequate fluorescence is found, a foreign substance is present, for example a bacterium, virus or other type of matter. Both lasers are directed onto a test probe and detections are made. Signals are produced and compared, and then the test signal is compared and reduced by the calibration factor. The remaining value is the final fluorescent value which indicates the presence or absence of foreign substances.

### FIELD OF THE INVENTION

This invention pertains to probe detection devices and particularly to a calibrating reader for reading probes that fluoresce in the presence of electromagnetic radiation of at least one particular frequency.

### BACKGROUND OF THE INVENTION

Most measuring instruments must be calibrated, and calibration is particularly important when measuring small quantities of almost any parameter. Even with the best calibration, changes in environmental conditions during a measurement could introduce significant errors. In the reading of a probe for a material, such as a biological agent, calibration is particularly important, and is particularly susceptible to error due to calibration problems and environmental changes. The present invention addresses these problems by the use of a technique that compensates for changing conditions and in a sense updates the calibration of the instrument. This is called self calibration in the context of this application.

### SUMMARY

The present invention provides an instrument and method for reading probes that detect a target substance such as bacteria. When the probes are exposed to the target substance and then illuminated by electromagnetic radiation of a specific frequency, the probes will fluoresce. To begin of the process of reading probes, a calibration process is performed first. The calibration is preferably performed using two matched lasers which are directed onto a non-fluorescing calibration probe and detecting the light eminating from each probe while being illuminated first by one laser and then the other. This procedure produces first and second calibration signals. A calibration factor is calculated by comparing the two calibration signals, and the calibration factor is stored and used each time a test probe is read. As mentioned above, a test probe will fluoresce when exposed to a target and will not fluoresce if it has not been exposed to the target. To read a test probe, it is illuminated with a first frequency of electromagnetic radiation, such as laser light, and the intensity of light emanating from the probe is detected producing a first test signal. Then, the probe is illuminated with a second frequency of electromagnetic radiation and the radiation eminating form the probe is detected producing a second test signal. The first frequency is chosen to not cause the probe to fluoresce uder any circumstances and the second frequency will cause the probe to fluoresce if the probe has been previously exposed to a target. The first test signal is compared to the second test signal to produce a comparison and the comparison is analyzed using the calibration factor to determine whether the test probe was fluorescing.

Preferably, the comparison of the first and second calibration detect signals is made by subtracting one from the other to produce the calibration factor, and likewise the comparison of the first and second test detect signals is made by subtracting one from the other to produce the comparison, which is referred to as the tested light difference. the calibration factor is then subtracted from the tested light difference to produce the final light value. Based on the final light value, a determination is made as to whether the probe was fluorescing to indicate the presence of the target. Preferably, if the final light value is below a threshold, there is a determination of no fluorescence. If the final light value is above the threshold, there is a determination that the proble was fluorescing and the target was present.

The method for calibration may be performed before every reading taken, which allows increased accuracy in readings. The slide reader reads slides on which are disposed a plurality of probes, which fluoresce when exposed to both a target substance and when illuminated by electromagnetic radiation having a first frequency. This fluorescence indicates the presence of a substance in the probe, the substance being, for example, an infectious disease. One embodiment of the instrument consists of a slide holder, a first electromagnetic radiation source, a second electromagnetic radiation source, a detector, and a data processor.

The slide holder holds a slide. The first electromagnetic radiation source illuminates the probes on the slide for a period of time with an electromagnetic radiation at the first frequency. The second electromagnetic radiation source illuminates the probes with electromagnetic radiation at a different frequency from the first frequency during a second time period different from the first time period. The detector detects electromagnetic radiation emanating from the probes while illuminated by the first and second electromagnetic radiation sources. The data processor is produces an analysis signal based on the detected electromagnetic radiation, which indicates the presence or absence of fluorescing electromagnetic radiation.

In one embodiment of the invention, the electromagnetic radiation sources are lasers, and in the preferred embodiment, those lasers are matched. Matched lasers are those which have substantially the same energy output but different frequencies. One embodiment of the instrument includes a beam splitter that splits both a first beam produced by the first electromagnetic radiation source and a second beam produced by the second electromagnetic radiation source into a transmitted beam and a reflected beam for both the first and second sources (resulting in four beams total). In the preferred embodiment, either the first transmitted beam and the second reflected beam or the second transmitted beam and the first reflected beam are transmitted toward the slide. The beam splitter is a 50% transmittance, 50% reflectance splitter in the ideal embodiment, so that the power of each beam striking the slide will be substantially the same. The ideal embodiment also comprises an instrument housing for reducing the amount of unwanted electromagnetic radiation in the instrument.

The preferred embodiment includes a source filter and a detector filter. The source filter is positioned so that the electromagnetic radiation sources are directed through the source filter. The filter is for filtering electromagnetic radiation having an unwanted frequency. The detector filter is placed so that the electromagnetic radiation entering the detector passes through the filter first. The detector filter also removes electromagnetic radiation having an unwanted frequency.

The present invention provides a method for calibrating the electromagnetic radiation sources for a slide reader that reads a plurality of probes on a slide. The direction of the first electromagnetic radiation beam is onto a non-fluorescing probe on the slide. The first electromagnetic radiation beam has a first frequency. The first detection occurs from the first electromagnetic probe radiation which is emanating from the probe while it is illuminated by the first electromagnetic radiation beam. The first electromagnetic radiation signal corresponds to the first electromagnetic probe radiation. The direction of a second electromagnetic radiation beam is also onto the non-fluorescing probe on the slide, and the second beam has a second frequency, which is different from the first frequency. The second detection occurs from the second electromagnetic probe radiation which is emanating from the probe while it is illuminated by the second electromagnetic radiation beam. The second electromagnetic radiation signal corresponds to the second electromagnetic probe radiation. The calibration factor is calculated by comparing the first and second electromagnetic radiation signals.

In one embodiment of the method, the first and second electromagnetic radiation signals are compared based on a difference method. The difference method consists of subtracting the second electromagnetic radiation signal from the first electromagnetic radiation signal which results in a calibration factor. Then a final fluorescence value is calculated. The first step is directing the first electromagnetic radiation beam onto a test probe on the slide. The test probe is a probe that is known not to fluoresce while illuminated by the first frequency. Next, a detector detects a first test electromagnetic radiation. The first test electromagnetic radiation emanates from the test probe while it is illuminated by the first electromagnetic radiation beam. A data processor then produces a first test signal. The first test signal corresponds to the first test electromagnetic radiation. The next step is directing the second electromagnetic radiation beam onto the test probe on the slide. The detector then detects a second test electromagnetic radiation. The second test electromagnetic radiation emanates from the test probe while it is illuminated by the second electromagnetic radiation beam. The data processor produces a second test signal that corresponds to the second test electromagnetic radiation. Then the final fluorescence value is calculated by a two step process. The first is subtracting the first and second test signals resulting in a test difference signal. The second is subtracting the calibration factor, which was calculated in the original method, from the test difference signal.

In another embodiment of the invention, the first and second electromagnetic radiation signals are compared based on a percentage method whereby the first and second electromagnetic radiation signals are compared. The percentage method consists of finding a ratio of the second electromagnetic radiation signal to the first electromagnetic radiation signal. A final fluorescence value is found by the following method. The first step is directing the first electromagnetic radiation beam onto a test probe on the slide. Next a detector detects a first test electromagnetic radiation. The first test electromagnetic radiation emanates from the test probe while it is illuminated by the first electromagnetic radiation beam. A data processor produces a first test signal that corresponds to the first test electromagnetic radiation. The next step is directing the second electromagnetic radiation beam onto the test probe on the slide. A detector then detects a second test electromagnetic radiation. The second test electromagnetic radiation emanates from the test probe while it is illuminated by the second electromagnetic radiation beam. The data processor produces a second test signal that corresponds to the second test electromagnetic radiation. Then the data processor calculates the final fluorescence value by a two step process. The data processor divides the second test signal by the first test signal resulting in a test ratio signal. Then, the data processor subtracts the calibration factor, found in the previous method, from the test ratio signal, which results in the final fluorescence value.

In another embodiment, the method for formulating a calibration factor is repeated using a second non-fluorescing probe on the slide. The data processor produces a second iteration output signal based on the comparison of two electromagnetic radiation signals similar to the one iteration process described above. The data processor compares the calibration factor from the first iteration to the second iteration calibration factor, and a refined calibration factor, which is more accurate, is calculated. In other embodiments, the calibration factor may be refined through numerous iterations of the calibration process described above.

### BRIEF DESCRIPTION OF THE DRAWINGS

The advantages and features of the invention may best be understood by reference to the following Detailed Description of preferred embodiments, when considered in conjunction with the drawings in which:

FIG. 1 is a rear view of a probe analyzer with the rear covering removed to reveal the analyzer construction;

FIG. 2 is a somewhat diagrammatic cross sectional view of a slide and probe showing the probe illuminated by one source;

FIG. 3 is a somewhat diagrammatic cross sectional view of a slide and probe showing the probe illuminated by a second source;

FIG. 4 is a somewhat diagrammatic cross sectional view of a slide and probe showing fluorescent radiation being emitted by a probe;

FIG. 5 is a somewhat diagrammatic cross sectional view of a slide and probe showing the probe being illuminated by a laser through a beam splitter;

FIG. 6 is a somewhat diagrammatic cross sectional view of a slide and probe showing the probe being illuminated by a laser reflected from a beam splitter;

FIG. 7 is a somewhat diagrammatic cross sectional view of a slide and probe showing a non-fluorescing sample, which reflects a beam produced by an electromagnetic radiation source resulting in a beam;

FIG. 8 illustrates a slide and probe using a laser and light filters to illuminate the probe;

FIG. 9 illustrates a slide with multiple probes and multiple detectors with one detector for each probe;

FIG. 10 illustrates a first electromagnetic radiation source 38 and a second electromagnetic radiation source 42 mounted on a wheel shaped chamber.

FIG. 11 illoustrate a cylindrical chamber with first and second electromagnetic radiation sources positioned near one another,

FIG. 12 illustrates trapezoidal mount for the sources of electromagnetic radiation;

FIGS. 13 and 14 illustrate an embodiment where the radiation sources are mounted on a cone shaped chamber;

FIG. 15 is an overhead view of the electromagnetic radiation source chamber inside the laser housing, which houses the first and second electromagnetic radiation sources;

FIGS. 16-20 are views of a spherical mount for the radiation sources.

### DETAILED DESCRIPTION OF THE DRAWINGS

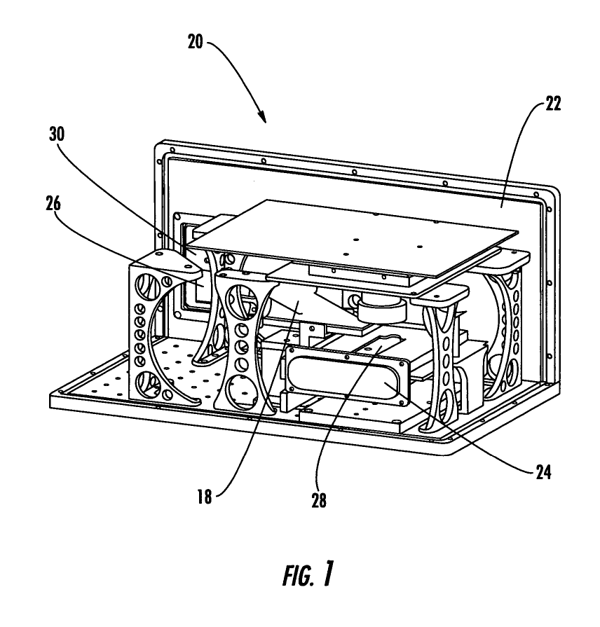

Referring now to the drawings in which like reference characters designate like or corresponding parts throughout the several views, there is shown in FIG. 1 a slide analyzer 20, including a housing 22 that is provided for containing the slide analyzer 20 and protecting the analyzer 20 from outside ambient electromagnetic radiation. A cassette 24 is mounted on the front surface of the housing 22 and moves into and out of the housing 22 carrying a slide 28, such as a slide for detecting biological entities. The operation of the analyzer 20 is controlled by a user through the keypad 26 and a display 30. The user enters commands and inquiries through the keypad 26 and receives information from the analyzer 20 through the display 30. The slide 28 is carried into the analyzer 20 by the cassette 24 in response to commands from the user through keypad 26. The slide 28 is typically a DNA micro-array. Typically, oligonucleotides are disposed on the slide 28 in a pattern or array. The oligonucleotides that are disposed on the slide 28 (or any other reactive agent that is placed on the slide 28) are referred to herein as probes. The laser housing 18 is disposed above and to the side of the slide 28 such that multiple lasers mounted in the laser housing may be directed onto the slide. FIGS. 5, 6, and 10-20 demonstrate some embodiments that may be used to mount the electromagnetic radiation sources 38 and 42 so that they will be in the intended line of sight 72 in order to strike the probe 34. The laser housing contains two lasers of varying wavelengths which have substantially identical power or energy outputs in an ideal embodiment.

Referring now to FIG. 2, the slide 28 has been prepared in advance before it is placed into the analyzer 20. For example, a slide may be prepared with a plurality of wells 32, shaped like craters, containing probes 34 with each probe 34 designed to bind with a specific type of material, such as a particular virus or bacteria or other type of matter. A material of interest, such as a food product, is then treated with fluorescent markers that attach to the sample food product and extraneous substances that may be present in the food products. For example, if bacteria are present in the food product, the fluorescent markers attach to the bacteria. The treated material of interest is then exposed to the slide 28. If one of the probes is designed to bind to salmonella bacteria, and if salmonella is present in the food product, the salmonella bacteria will bind to that particular probe 34 of the slide 28. The salmonella bacteria will carry the fluorescent markers and, thus, the presence of the salmonella on the probe may be detected by detecting florescent electromagnetic radiation from the florescent markers on the salmonella, which is bound to the probe 34. A common feature of each slide 28 is that it includes a plurality of spots that will produce electromagnetic radiation and thereby provide information.

A side view of a slide 28 with a well 32 containing a probe 34 cut out of the slide 28 is shown in FIG. 2. A detector 36 is positioned directly above the probe 34. The electromagnetic radiation sources 38 and 42 are shown very close in relation to the slide 28 and the probe 34. In reality they are much farther away from the slide 28 and the detector 36 but are shown close in the figures to facilitate effective displays. Also, the well 32 and probe 34 appear very large, but in reality are relatively small compared to the size of the sources 38 and 42. Again, this is for ease in demonstration purposes. A first electromagnetic radiation source 38 is positioned to shine a directed electromagnetic radiation 40 with known wavelength onto the probe 34. This directed electromagnetic radiation 40 should be known not to cause the sample or the fluorescent marker in the probe 34 to fluoresce.

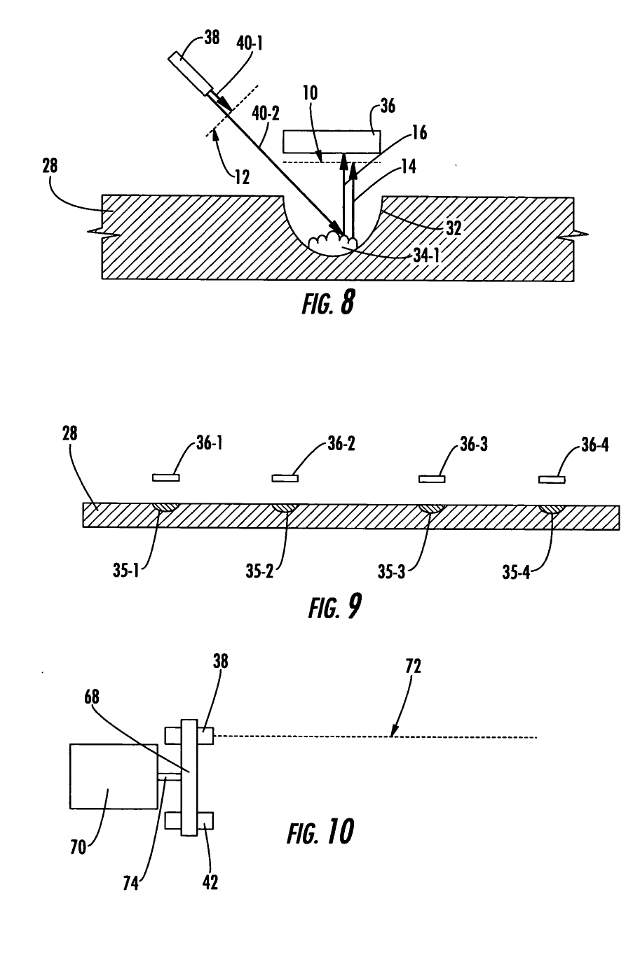

This result may be helped by using frequency filters such as the filters portrayed in FIG. 8. The source filter 12 eliminates all electromagnetic radiation leaving the source and thus going onto the probe with an unwanted wavelength. The detector filter 10 filters out all wavelengths known to be interference from the detector. Beam 40-2 has a wavelength known to cause fluorescence of a probe treated with a certain substance, and the source filter 12 allows it to pass. However, beam 40-1 does not have the intended wavelength and the source filter 12 does not allow it pass and strike the probe. Similarly, the detector filter 10 allows beam 16 to pass, which is in the known fluorescing range, whereas the detector filter 10 does not allow beam 14 to pass, which has a wavelength known not to be in the fluorescing range. Beam 14 may represent any type of interfering electromagnetic radiation. Although these filters are not present in any other drawings, in an ideal embodiment of the invention, both filters would be present. However, to reduce the clutter of the other figures, the filters have been left out. Various filters may be used, for example a different filter may be used when different electromagnetic radiation sources are active, or different filters may be used for a calibration mode as opposed to a test mode. Also, in the ideal embodiment of the analyzer, the electromagnetic radiation sources may be left on while readings are taken, and the filters would be designed to filter the electromagnetic radiation source frequencies out of the measured result.

FIG. 7 illustrates a non-fluorescing sample 34-1, which reflects the beam 40 produced by the electromagnetic radiation source 38 resulting in beam 58. This illustration demonstrates that the probe is not being energized by the electromagnetic radiation source and will not result in fluorescence, however it is not intended to indicate that the entire electromagnetic radiation is reflected or scattered in practice. Referring simultaneously to FIG. 4, the detector 36 measures the fluorescing electromagnetic radiation 46 coming off the probe 34-1. The incident electromagnetic radiation 46 read by the detector 36 while the first source 38 is illuminating the probe represents an offset value, that is, the amount of ambient 64 and non-fluorescing electromagnetic radiation 58 and 62 that is a result of the first electromagnetic radiation source 38. FIG. 7 illustrates ambient electromagnetic radiation 64 reflecting off of the probe, beam 66. The amount of ambient electromagnetic radiation, however, would be infinitesimal given the electromagnetic radiation proof housing 22 of the apparatus 20. Another source of noise could be the scattering of the first electromagnetic radiation source, represented by beam 61. The reflection of the electromagnetic radiation source from the probe 34-1 is represented by beam 60 and the reflection of beam 60 from the well 32 is represented by beam 62, both of which could also be sources of noise.

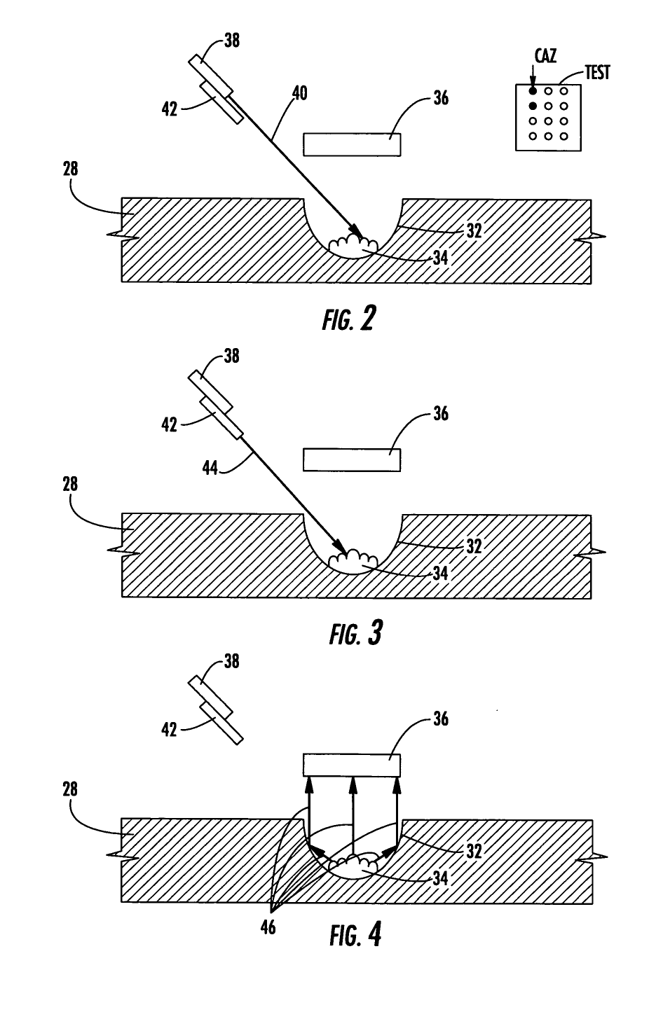

Next, as shown in FIG. 3, a second electromagnetic radiation source 42 is positioned to shine a second directed electromagnetic radiation 44 onto the probe 34. The second electromagnetic radiation source 42 has a wavelength known to make the fluorescent marker but not the sample in the probe 34 fluoresce. In FIGS. 2-4 and 9 the electromagnetic radiation sources 38 and 42 are shown side by side and offset from each other. This portrayal is intended to indicate that the electromagnetic radiation sources are directed from the same position; this portrayal is used so that both sources 38 and 42 may be easily shown on the same figure. The embodiments of FIGS. 5, 6, and 10-20 could be used to achieve the result of sources directed from the same position or a similar result. Ambient electromagnetic radiation, as well as scattering and reflecting electromagnetic radiation from the source, which are illustrated in FIG. 7, may still be present when the second beam 44 is directed on the known-to-fluoresce probe 34-2. The electromagnetic radiation, which may still be represented by 46 in FIG. 4, is measured by the detector 36. This reading represents the fluorescence of the tag during the second electromagnetic radiation source illumination. This value should include all of the noise contained in the first reading, as the sources were positioned similarly. The readings may be compared either by a difference method, by a percentage method, or by any other method which takes into account the power or energy relationship between the two sources. This relationship may then be applied to the test readings to eliminate noise in the test readings. This is the basic process used for calibration of the analyzer, but many variations on this basic process may be used such as different normalization algorithms, various electromagnetic radiation sources, different types of biological slides, the filters discussed above, etc. The embodiments described herein are not meant to limit the scope of the invention, but merely to provide examples of how the invention may be implemented.

In one embodiment of the normalization algorithm for reducing noise in the signal produced by fluorescing probes, a difference method is utilized. The difference method is most effective in a situation where the ratio of the first laser readings from probe to probe on the same slide remain close to one while the ratio of the second laser readings from probe to probe differ greatly. The difference method for normalization is basically taking a first difference between two laser measurements which represents the power or energy difference between the lasers and is generally considered constant. Then that value is subtracted from the measured test difference between the same two lasers on a test probe, and the remaining value is the total signal minus the noise value resulting in the pure signal. This is a short explanation of the difference method so that the following example may be understood; the full description of the difference method follows the example.

In the following example the values are not intended to represent an actual situation and are given for demonstration of a situation where the difference method would be effective. The first step is to find a calibration factor. That is done by directing two lasers onto the same probe and calculating the difference in the readings of the fluorescing electromagnetic radiation after each laser is turned off. For this example assume that the measurements (first reading, second reading) were (1.0, 1.1). In FIG. 9, the first probe 35-1 on a slide 28 is energized by two lasers 38 and 42 independently, the first resulting in a reading of (1.0) and the second resulting in (1.1). Similar measurements are taken on three other probes, 35-2, 35-3, and 35-4, on the same slide 28 with the same lasers 38 and 42 resulting in a second probe 35-2 reading of (1.0, 1.2), a third probe 35-3 reading of (1.0, 1.3), and a fourth probe 35-4 reading of (1.0, 1.4). From these measurements, each probe to probe ratio of the first laser 38 measurements is one, while the ratio of the second laser 42 measurements is greater than one. A constant first laser reading, such as the first reading in this example indicates a uniform slide. In this situation, either the difference or the percentage method (described below) would probably be effective. However, if the situation were one in which the slide was known to differ greatly from probe to probe, then the difference method would reduce error that the percentage method compounded. Thus, the difference method, which will take into account the slide properties without skewing the results, should be used in a slide with non-uniform properties as opposed to the percentage method, which may skew the results of the analysis by inflating the calculated final electromagnetic radiation value. Therefore, it will be helpful to have a thorough knowledge of the tendencies of the slides being used in the analysis process. The flexibility of using various techniques for finding foreign substances may reduce the production costs of slides because they do not have to meet strict uniformity across an entire slide, especially when using the difference method for calculation.

Following is a more detailed description of the difference method. A calibration factor may be calculated by subtracting the reading resulting from the first electromagnetic radiation source from the reading resulting from the second electromagnetic radiation source. This value will serve as normalization for removing noise based on readings that are not the result of fluorescence of a marker. Typically, this value is considered constant and stable, but multiple iterations of the above procedure may result in a more accurate calibration factor.

The next step is to repeat the process described above using the same electromagnetic radiation sources 38 and 42 to calculate a test difference signal. Once again, the first electromagnetic radiation source 38 is shown onto a probe 34, but this time the probe is different, it is a test probe. The first electromagnetic radiation source 38 is known not to cause the sample or the fluorescent tag to fluoresce in the probe 34, as shown in FIG. 7. The second electromagnetic radiation source 42 is known not to cause the sample to fluoresce as well. However, the second electromagnetic radiation source 42 is unknown whether to cause the fluorescent tag in the sample to fluoresce. The first electromagnetic radiation source 38 is directed onto the probe just as above and a reading is taken. Next, the second electromagnetic radiation source 42 is directed onto the probe just as above and a second reading is taken. The first value is subtracted from the second value to obtain the test difference signal. This calculation is generally considered accurate, but to obtain a more accurate calculation, many iterations of the procedure may be performed. Once the test difference signal is calculated, the final fluorescence signal may be calculated by subtracting the calibration factor from the test difference signal. This is the fluorescent electromagnetic radiation value of interest for each test. This value indicates what fluorescent marker is present which has bonded to a foreign substance in a sample thus indicating the presence of that foreign substance in the sample. The calibration factor is an offset used to remove any unwanted electromagnetic radiation from the readings of the test difference signal, thus when it is subtracted from the test difference signal, the useful fluorescent value is obtained.

Another embodiment of the normalization used to reduce noise is the percentage method. The percentage method is most effective in a situation where the ratio of the first laser readings from probe to probe on the same slide differ proportionally to the ratio of the second laser readings from probe to probe. The percentage method for normalization is basically taking a fluorescent reading resulting from two lasers and calculating a percentage relationship between the two readings which represents a power or energy difference. That percentage is then used to normalize the remaining test probe readings using the same lasers. This is a short explanation of the percentage method intended to facilitate understanding of the following example; a detailed description of the percentage method is below.

In the following example the values are not intended to represent an actual situation and are given merely for demonstration of a situation where the percentage method would be effective, just as the difference method example. The normalization step is performed on a probe 34-1 that is known to result in fluorescence by neither of the wavelengths of the electromagnetic radiation sources 38 and 42. For the example assume that the ratio of second reading to first reading is: (1.1/1.0). Thus, the ratio is (1.1) or in other words the second reading is (110%) of the first reading. This value represents the normalization or calibration factor between the two sources. In FIG. 9, the first probe 35-1 on a slide 28 is energized by two lasers 38 and 42 independently, the first resulting in a reading of (1.0) and the second resulting in (1.1). Similar measurements are taken on three other probes, 35-2, 35-3, and 35-4, on the same slide 28 with the same lasers 38 and 42 resulting in a second probe 35-2 reading in the form (second reading, first reading) of (2.2., 2.0), a third probe 35-3 reading of (3.3, 3.0), and a fourth probe 35-4 reading of (4.4, 4.0). Each of these readings yields a calculated ratio where the second reading is (110%) of the first reading, therefore no fluorescence is present in any of the four probes according to the percentage method. As seen in the readings, the ratios of the first laser readings are increasing proportionally to the ratios of the second laser readings. From probe 35-1 to 35-2 the increase of both is by a factor of two. From 35-1 to 35-3 the increase is by a factor of three and by a factor of four with probe 35-4. This situation is best analyzed using the percentage method because the characteristics of the slide may be causing the proportional increase of electromagnetic radiation readings for both lasers and thus a difference method would skew the results (it would indicate increasing fluorescence from probes 35-2 to 35-3 to 35-4).

Following is a detailed explanation of the percentage method. A calibration factor between the two sources may be calculated by first calculating a ratio of the reading resulting from the second electromagnetic radiation source to the reading resulting from the first electromagnetic radiation source. This value will serve as a normalization for removing noise based on incident electromagnetic radiation readings that are not the result of fluorescence of a marker. Typically, this value is considered constant and stable, but multiple iterations of the above procedure may result in a more accurate percentage relationship.

The next step is to modify the process described above using the same electromagnetic radiation sources 38 and 42 to calculate a ratio test signal. Once again, the first electromagnetic radiation source 38 is directed onto a test probe 34. The first electromagnetic radiation source 38 is known not to cause the sample or the fluorescent tag to fluoresce in the probe 34, as shown in FIG. 7. The second electromagnetic radiation source 42 is known not to cause the sample to fluoresce as well. However, the second electromagnetic radiation source 42 is unknown whether to cause the fluorescent tag in the sample to fluoresce. The first electromagnetic radiation source 38 is directed onto the probe just as above and a reading is taken. Next, the second electromagnetic radiation source 42 is directed onto the probe just as above and a second reading is taken. The ratio of the second value to the first value is taken, and this is referred to as the test ratio signal. This calculation is generally considered accurate, but to obtain a more accurate calculation, many iterations of the procedure may be performed. Once the test ratio signal is calculated, this value is compared to the calibration factor. If the test ratio signal is higher than the calibration factor then the probe is fluorescing. The calibration factor may be subtracted from the test ratio signal to determine what percentage of fluorescence is present. This percentage could be referred to as the final fluorescence signal, and the final fluorescence signal may be compared to a list of final fluorescence signals representing foreign bodies possibly present on the slide.

In one embodiment of the analyzer, the electromagnetic radiation sources are lasers which are directed from the same location. FIGS. 2, 3, and 4 include electromagnetic radiation sources, 38 and 42, which appear side by side. If the reading correlating to the first electromagnetic radiation source 38 is taken and the first electromagnetic radiation source is removed and the second electromagnetic radiation source 42 is moved into the same position that the first electromagnetic radiation source 38 was directed, the results of the procedure may be more accurate than if the lasers were directed from different locations. In other words, the electromagnetic radiation sources should be directed from the same location to reduce potential error. This can be achieved through the embodiment shown in FIG. 10. In FIG. 10, the first electromagnetic radiation source 38 is positioned so that it will direct its beam along the target line of sight 72 that intersects with the intended probe. Both the first electromagnetic radiation source 38 and the second electromagnetic radiation source 42 are mounted on a wheel shaped chamber 68 similar to the chamber of a revolving firearm. In FIG. 10 the two sources 38 and 42 are mounted opposite each other on the chamber 68, that is they are one hundred and eighty degrees apart. A controllable rotation device such as a stepping motor 70 turns a shaft 74 which is connected to the chamber 68 in order to rotate the electromagnetic radiation sources into position with the intended line of sight 72. In other possible embodiments, the chamber 68 could be many other shapes and the electromagnetic radiation sources could be mounted in many different positions on any of those shapes.

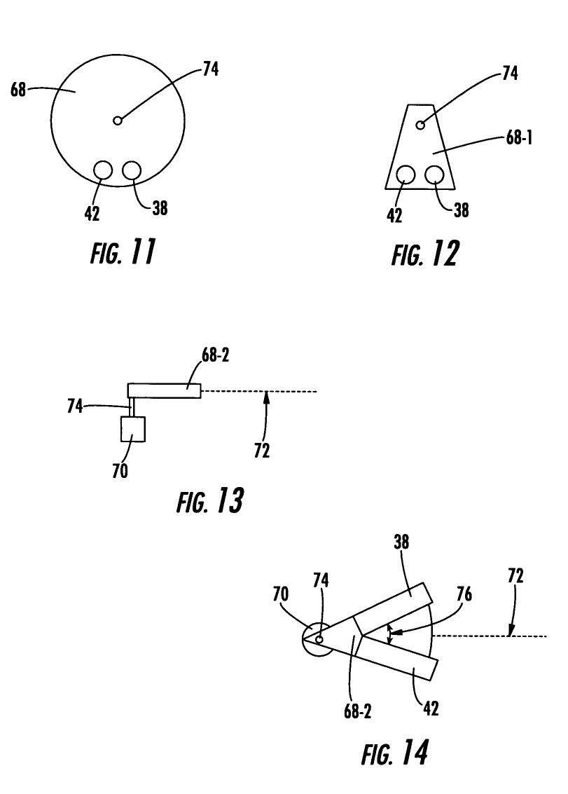

For example, in FIG. 11 a cross section of a cylindrical chamber is shown with the first and second electromagnetic radiation sources 38 and 42 positioned near one another in order to reduce the amount of time between when the first electromagnetic radiation source 38 and when the second electromagnetic radiation source 42 correlate respectively with the intended line of sight 72, which is out of the paper in FIG. 11. In another embodiment, FIG. 12 illustrates a similar design as FIG. 11 except that the chamber 68-1 consists of much less material than a cylindrical chamber 68. In this case space and possibly energy could be better conserved by reduced area and weight.

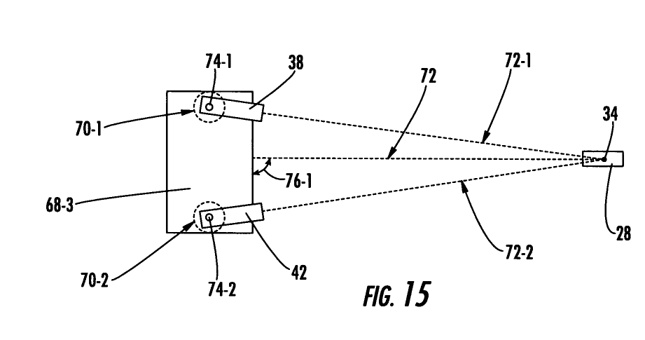

FIG. 13 and 14 illustrate yet another embodiment of the apparatus. The shaft 74 of the stepper motor 70 is connected to a cone shaped chamber 68-2 on which both the first electromagnetic radiation source 38 and the second electromagnetic radiation source 42 are mounted. In this case the major axis of the shaft 74 is perpendicular to the major axis of the electromagnetic radiation sources 38 and 42 as opposed to the embodiments illustrated in FIGS. 10, 11, and 12 where the major axis of the shaft 74 is parallel to the major axis of the electromagnetic radiation sources 38 and 42. Referring again to FIGS. 13 and 14, the stepper motor 70 can accurately step between the two positions necessary to position each of the electromagnetic radiation sources 38 and 42 into the intended line of sight 72. This embodiment may be preferred over the previously discussed embodiments because the angle 76 between the first and second electromagnetic radiation sources 38 and 42 may be minimal thus allowing the electromagnetic radiation sources to be positioned more easily than in other embodiments.

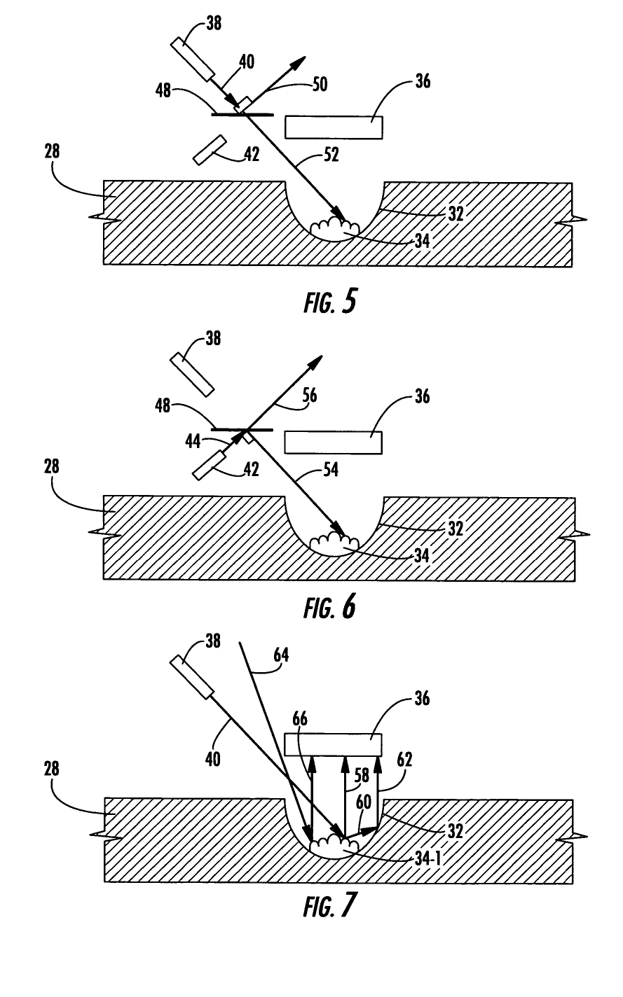

In another embodiment, shown in FIG. 5, a beam splitter 48 is used to ensure that the electromagnetic radiation sources, 38 and 42, are directed from the same position. This embodiment would be preferred over the mechanical embodiments detailed above because it would conserve both time and energy. Ideally, a one-half transmittance, one-half reflectance beam splitter 48 would be used with electromagnetic radiation sources 38 and 42 directed at forty-five degree angles to the beam splitter 48. If the lens used as the beam splitter was imperfect or improperly placed, and the problem remained constant, the results of the calibration would be accurate regardless of the imperfection. For instance, if the beam splitter 48 actually allowed forty-eight percent transmittance and fifty-two percent reflectance and the lens was the same for both electromagnetic radiation sources, the resulting readings would be accurate regardless of the imperfect lens everything else remaining constant. This embodiment is shown in FIGS. 5 and 6. FIG. 5 shows the first electromagnetic radiation source 38 being directed onto the beam splitter 48 at a forty-five degree angle. The first reflection beam 50 is reflected from the beam splitter 48 at a ninety degree angle to the first electromagnetic radiation source 40. Ideally, beam 50 corresponds to fifty percent of the power of beam 40. The first transmittance beam 52 is transmitted from the beam splitter 48 at a zero degree angle to the first electromagnetic radiation source 40. Beam 52 is directed onto the probe 34. The first electromagnetic radiation source 38 is removed and the detector 36 reads the incident electromagnetic radiation 46 just as above. Next, the second electromagnetic radiation source 42 directs a electromagnetic radiation 44 onto the beam splitter 48 at a forty-five degree angle as shown in FIG. 6. The second reflected beam 54 is reflected ideally at a ninety degree angle to beam 44. Beam 54 is directed onto the probe 34 and ideally represents fifty percent of the power of beam 44. The second transmitted beam 56 is transmitted through the beam splitter 48 ideally at a zero degree angle to beam 44. The second electromagnetic radiation source 42 is removed and the detector 36 reads the incident electromagnetic radiation 46 just as above. The remaining procedure for finding a calibration factor is similar.

FIG. 15 is an overhead view of the electromagnetic radiation source chamber 68-3, inside the laser housing 18, which houses both the first electromagnetic radiation source 38 and the second electromagnetic radiation source 42. The electromagnetic radiation sources are mounted respectively on a first stepper motor shaft 74-1 and a second stepper motor shaft 74-2. The shafts 74-1 and 74-2 are connected to the first stepper motor 70-1 and the second stepper motor 70-2 respectively. This embodiment is different than those above because the electromagnetic radiation sources are not directing their electromagnetic radiation along the intended line of sight 72, but they are directing beams along a first path 72-1 and a second path 72-2. Both the first path 72-1 and the second path 72-2 intersect with the probe 34 disposed on the slide 28. Despite the varying directions that the electromagnetic radiation sources are directed, the readings will remain accurate as long as the distance from the chamber 68-3 to the probe 34 and the angle 76-1 between the intended line of sight 72 and the chamber 68-3 remain constant. The stepper motors 70-1 and 70-2 may be used to readjust the electromagnetic radiation sources 38 and 42 so that the first and second paths 72-1 and 72-2 represent the electromagnetic radiation source direction and intersect the probe 34. This embodiment provides the means to reduce the amount of mechanical movement necessary while keeping maximum individual laser power.

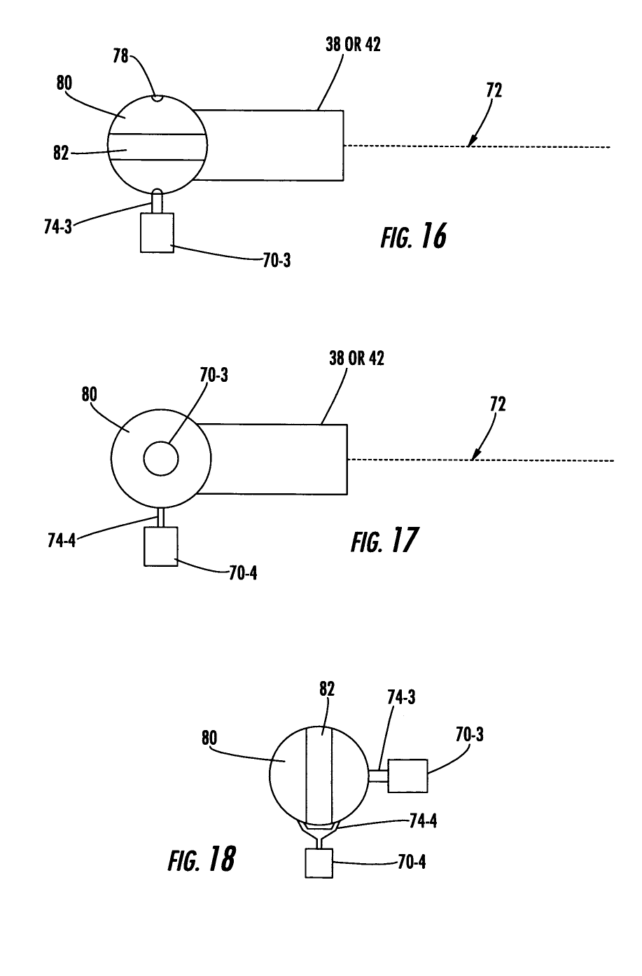

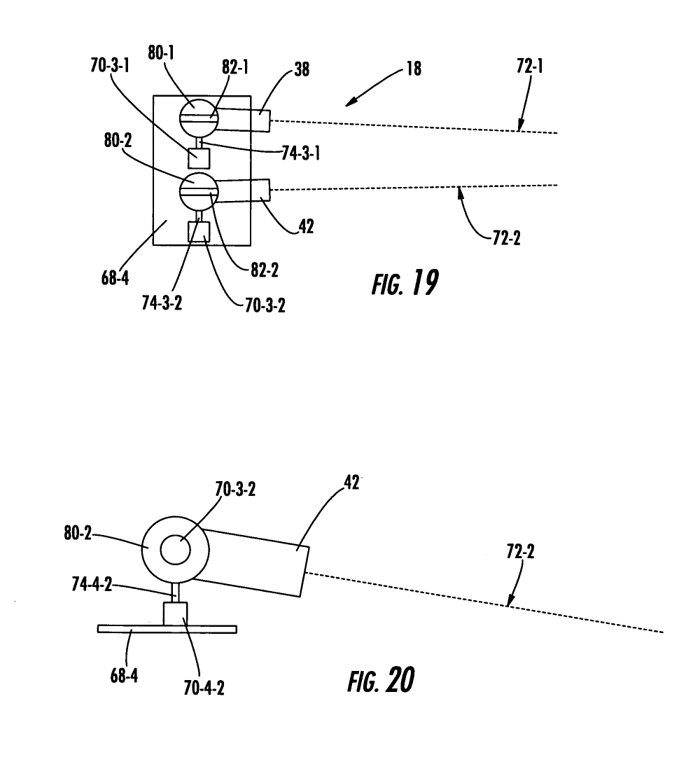

Moving to another embodiment of the electromagnetic radiation source mechanism, FIG. 16 illustrates a device that would allow for both vertical and horizontal adjustment of the electromagnetic radiation source. This would compensate for the range of motion necessary to test multiple probes on a slide. FIG. 16 is a top view of the device which has a spherical mount 80 connected to one of the electromagnetic radiation sources 38 or 42 by means of a rotating cylinder 82 connected to a vertical motion drive shaft 74-3 which passes through the center of the spherical mount 80 to the opposite side, represented by 78. The vertical motion drive shaft is connected to a vertical stepping motor 70-3, which will allow the electromagnetic radiation source to be directed to an accurate vertical coordinate to coincide with the intended line of sight 72. FIG. 17 is a side view showing a horizontal stepping motor 704, which is connected to a horizontal drive shaft 74-4. The horizontal drive shaft is connected to the spherical mount 80 without interfering with the motion of the rotating cylinder which provides for vertical placement of the electromagnetic radiation source 38 or 42. This connection is illustrated in FIG. 18, which is a view from behind the device or looking in the same direction as the electromagnetic radiation source is directed. In this view both the vertical stepping motor 70-3 and the horizontal stepping motor 70-4 are in sight. The horizontal drive shaft 74-4 from the horizontal stepping motor is shown not to touch the rotating cylinder 82. This dual drive device allows for both vertical and horizontal direction of the electromagnetic radiation source. FIG. 19 demonstrates an entire electromagnetic radiation source housing device 18 similar to the one in FIG. 15. FIG. 19 is a top view of a dual source configuration using the device illustrated in FIGS. 16-18. In this embodiment the horizontal stepping motors 70-4-1 and 70-4-2, which cannot be seen because they are hidden by the spherical mounts 80-1 and 80-2, are mounted onto the chamber 68-4. Thus both electromagnetic radiation sources 38 and 42 may be directed onto a single probe. FIG. 20 illustrates a side view of the dual source configuration of FIG. 19 showing the second horizontal stepping motor 70-4-2. The first electromagnetic radiation source 38 and its drive mechanism are behind the second electromagnetic radiation source 42 and are thus not shown.

In the preferred embodiment of the device, two matched lasers are used as electromagnetic radiation sources 38 and 42. The description “matched” means that the lasers have substantially the same power or energy output while having distinct, yet similar, wavelengths. Because the lasers are matched, a known incident electromagnetic radiation difference between the two lasers will be more accurate and constant than two unmatched lasers. In addition, if the lasers are matched, their difference in energy is very small compared to a fluorescent signal. There would be less chance of error if the power difference is very small when compared to the signal strength. A calibration factor process could be reiterated to obtain a truly accurate calibration factor and then multiple tests could be performed using the two matched lasers without the need to find a new calibration factor.

In the above Description, a number of exemplary embodiments are described, and It will be understood that the invention is capable of numerous modifications, rearrangements and substitutions of parts without departing from the scope of the invention as defined in the claims.

1. An instrument for reading slides, each slide having a plurality of probes, each probe for fluorescing when both exposed to a target and illuminated by electromagnetic radiation of a first frequency comprising:
a. A slide holder for holding a slide,
b. A first electromagnetic radiation source for illuminating the probes at one or more first time intervals with an electromagnetic radiation at the first frequency,
c. A second electromagnetic radiation source for illuminating the probes with electromagnetic radiation at a second frequency at one or more second time intervals, the second time intervals and second frequency being different from the first time intervals and first frequency,
d. A detector for detecting electromagnetic radiation emanating from the probes, and
e. A data processor for producing an analysis signal based on the detected electromagnetic radiation indicating the presence or absence of fluorescing electromagnetic radiation.

2. The instrument of claim 1, wherein the first and second electromagnetic radiation sources are lasers.

3. The instrument of claim 1, wherein the first and second electromagnetic radiation sources are matched lasers producing substantially the same energy output.

4. The instrument of claim 1, wherein:
a. The first electromagnetic radiation source produces a first beam traveling along a first beam path,
b. The second electromagnetic radiation source produces a second beam traveling along a second beam path,
c. The first beam and the second beam are directed toward a beam splitter, the beam splitter for splitting the first beam into a first transmitted beam and a first reflected beam and for splitting the second beam into a second transmitted beam and a second reflected beam,
d. The first beam path is identical to a first path traveled by at least one of the first transmitted beam and the first reflected beam, and
e. The second beam path is identical to a second path traveled by at least one of the second transmitted beam and the second reflected beam.

5. The instrument of claim 1, further comprising an instrument housing for removing unwanted electromagnetic radiation from the instrument.

6. The instrument of claim 1, further comprising:
a. A source filter disposed such that the electromagnetic radiation sources are directed through the source filter, the source filter for filtering electromagnetic radiation having an unwanted frequency and
b. A detector filter disposed such that electromagnetic radiation entering the detector passes through the detector filter, the detector filter for filtering electromagnetic radiation having an unwanted frequency.

7. A method for calibrating electromagnetic radiation sources for a slide reader that reads a plurality of probes on a slide which comprises:
a. Directing a first electromagnetic radiation beam having a first frequency onto a non-fluorescing probe on the slide,
b. Detecting a first electromagnetic probe radiation emanating from the probe while illuminated by the first electromagnetic radiation beam,
c. Producing a first electromagnetic radiation signal corresponding to the first electromagnetic probe radiation,
d. Directing a second electromagnetic radiation beam having a second frequency, the second frequency being different from the first frequency, onto the non-fluorescing probe on the slide,
e. Detecting a second electromagnetic probe radiation emanating from the probe while illuminated by the second electromagnetic radiation beam,
f. Producing a second electromagnetic radiation signal corresponding to the second electromagnetic probe radiation,
g. Comparing the first and second electromagnetic radiation signals, and
h. Calculating a calibration factor based on the comparison.

8. The method of claim 7, wherein the first and second electromagnetic radiation signals are compared based on a difference method.

9. The method of claim 7, wherein the first and second electromagnetic radiation signals are compared based on a difference method of subtracting the second electromagnetic radiation signal from the first electromagnetic radiation signal.

10. The method of claim 7, wherein the first and second electromagnetic radiation signals are compared and the calibration factor is calculated based on a difference method of subtracting the second electromagnetic radiation signal from the first electromagnetic radiation signal whereby a final fluorescence signal is obtained by:
a. Directing the first electromagnetic radiation beam onto a test probe on the slide, the test probe known not to fluoresce while illuminated by the first frequency of the first electromagnetic radiation beam,
b. Detecting a first test electromagnetic radiation emanating from the test probe while illuminated by the first electromagnetic radiation beam,
c. Producing a first test signal corresponding to the first test electromagnetic radiation,
d. Directing the second electromagnetic radiation beam onto the test probe on the slide,
e. Detecting a second test electromagnetic radiation emanating from the test probe while illuminated by the second electromagnetic radiation beam,
f. Producing a second test signal corresponding to the second test electromagnetic radiation,
g. Subtracting the first and second test signals resulting in a test difference signal, and
h. Subtracting the calibration factor from the test difference signal resulting in the final fluorescence signal.

11. The method of claim 7, wherein the first and second electromagnetic radiation signals are compared based on a percentage method.

12. The method of claim 7, wherein the first and second electromagnetic radiation signals are compared based on a percentage method of finding a ratio of the second electromagnetic radiation signal to the first electromagnetic radiation signal resulting in a calibration factor.

13. The method of claim 7, wherein the first and second electromagnetic radiation signals are compared and the calibration factor is calculated based on a percentage method of finding a ratio of the second electromagnetic radiation signal to the first electromagnetic radiation signal whereby a final fluorescence signal is obtained by:
a. Directing the first electromagnetic radiation beam onto a test probe on the slide, the test probe known not to fluoresce while illuminated by the first frequency of the first electromagnetic radiation beam,
b. Detecting a first test electromagnetic radiation emanating from the test probe while illuminated by the first electromagnetic radiation beam,
c. Producing a first test signal corresponding to the first test electromagnetic radiation,
d. Directing the second electromagnetic radiation beam onto the test probe on the slide,
e. Detecting a second test electromagnetic radiation emanating from the test probe while illuminated by the second electromagnetic radiation beam,
f. Producing a second test signal corresponding to the second test electromagnetic radiation,
g. Dividing the second test signal by the first test signal resulting in a test ratio signal, and
h. Subtracting the calibration factor from the test ratio signal resulting in a final fluorescence signal.

14. The method of claim 7, wherein the first electromagnetic radiation beam and the second electromagnetic radiation beam are lasers.

15. The method of claim 7, wherein the first electromagnetic radiation beam and the second electromagnetic radiation beam are matched lasers, the first and second electromagnetic radiation beams producing substantially the same energy and the first and second frequencies being distinct.

16. The method of claim 7, further comprising:
a. Splitting the first electromagnetic radiation beam into a first transmitted beam and a first reflected beam at least one of which is directed onto the slide and
b. Splitting the second electromagnetic radiation beam into a second transmitted beam and a second reflected beam at least one of which is directed onto the slide.

17. The method of claim 7, further comprising:
a. Filtering the first electromagnetic radiation beam and the second electromagnetic radiation beam to remove unwanted frequencies and
b. Filtering the first electromagnetic radiation and the second electromagnetic radiation emanating from the probe to remove unwanted frequencies.

18. The method of claim 7, further comprising:
a. Directing the first electromagnetic radiation beam onto a second non-fluorescing probe on the slide,
b. Detecting a second iteration first electromagnetic radiation emanating from the probe while illuminated by the first electromagnetic radiation beam,
c. Producing a second iteration first electromagnetic radiation signal corresponding to the second iteration first electromagnetic radiation,
d. Directing the second electromagnetic radiation beam onto the second non-fluorescing probe on the slide,
e. Detecting a second iteration second electromagnetic radiation emanating from the probe while illuminated by the second electromagnetic radiation beam,
f. Producing a second iteration second electromagnetic radiation signal corresponding to the second iteration second electromagnetic radiation,
g. Comparing the second iteration first and second electromagnetic radiation signals,
h. Producing a second iteration output signal based on the comparison,
i. Comparing the second iteration output signal to the output signal, and
j. Producing a refined output signal based on the comparison of the second iteration output signal to the output signal.

19. A method for calibrating matched lasers for a slide reader that reads a plurality of probes on a slide which comprises:
a. Directing a first laser having a first frequency and a first energy output onto one of a plurality of non-fluorescing probes on the slide,
b. Detecting a first electromagnetic radiation emanating from the probe while illuminated by the first laser,
c. Producing a first electromagnetic radiation signal corresponding to the first electromagnetic radiation,
d. Directing a second laser having a second frequency, the second frequency being different from the first frequency, and a second energy output, the second energy output being substantially the same as the first energy output, onto the non-fluorescing probe on the slide,
e. Detecting a second electromagnetic radiation emanating form the probe while illuminated by the second laser,
f. Producing a second electromagnetic radiation signal corresponding to the second electromagnetic radiation,
g. Comparing the first and second electromagnetic radiation signals,
h. Producing an output signal based on the comparison,
i. Directing the first electromagnetic radiation beam onto a second non-fluorescing probe on the slide,
j. Detecting a second iteration first electromagnetic radiation emanating from the probe while illuminated by the first laser,
k. Producing a second iteration first electromagnetic radiation signal corresponding to the second iteration first electromagnetic radiation,
l. Directing the second electromagnetic radiation beam onto the second non-fluorescing probe on the slide,
m. Detecting a second iteration second electromagnetic radiation emanating from the probe while illuminated by the second laser,
n. Producing a second iteration second electromagnetic radiation signal corresponding to the second iteration second electromagnetic radiation,
o. Comparing the second iteration first and second electromagnetic radiation signals,
p. Producing a second iteration output signal based on the comparison,
q. Comparing the second iteration output signal to the output signal, and
r. Producing a refined output signal based on the comparison of the second iteration output signal to the output signal.

20. The method of claim 19, wherein:
a. The first and second electromagnetic radiation signals are compared based on either a difference method or a percentage method, the difference method of subtracting the second electromagnetic radiation signal from the first electromagnetic radiation signal resulting in a normalization signal whereby a final fluorescence signal is obtained, the percentage method of finding a ratio of the second electromagnetic radiation signal to the first electromagnetic radiation signal resulting in the normalization signal whereby the final fluorescence signal is obtained and
b. The second iteration first and second electromagnetic radiation signals are compared based on either the difference method or the percentage method.
